(19) United States
(12) Patent Application Publication
(10) Pub. No.: US 2025/0312545 A1
(43) Pub. Date: Oct. 9, 2025

(54) NO supply apparatus with automatic modification of alarms upon changes in dose

(71) Applicant: INOSYSTEMS, Antony (FR)

(72) Inventors: Yann BLANDIN, Antony (FR); Frederic MARCHAL, Antony (FR); Nathan PROUVEZ, Antony (FR); Nicolas TIRILLY, Antony (FR)

(21) Appl. No.: 19/083,432

(22) Filed: Mar. 19, 2025

(30) Foreign Application Priority Data
Apr. 4, 2024 (FR) 2403481

(51) Int. Cl. A61M 16/00; A61M 16/10; A61M 16/12

(52) U.S. Cl. CPC A61M 16/0051; A61M 16/1005; A61M 16/12; A61M 2016/0033; A61M 2202/0275; A61M 2205/18; A61M 2205/505; A61M 2205/581; A61M 2205/583

(57) ABSTRACT

The invention relates to an apparatus (1) for delivering NO-containing gas, comprising means for setting a dose of NO (50, 51) to make it possible to set a first dose of NO, and operating means (210) with a microprocessor (211), for determining on the basis of the first dose of NO set, first upper and lower alarm thresholds corresponding to first maximum and minimum NO contents. Means for modifying a dose of NO (50, 51) making it possible to modify or adjust the first dose of NO so as to obtain or fix a second dose of NO that is different to the first dose of NO. The operating means (210) are configured to automatically determine, on the basis of the second dose of NO, second upper and lower NO alarm thresholds that are different to the first upper and lower NO alarm thresholds. Installation (100) for supplying NO-containing gas, comprising such an NO delivery apparatus (1).

### TECHNICAL FIELD

The invention relates to an NO delivery apparatus comprising means for automatic modification of the alarms upon changes in NO dose, and to an installation for supplying an NO-based gas mixture to a patient, typically an NO/nitrogen (N₂) mixture, comprising such an NO delivery apparatus and a medical ventilator delivering a respiratory gas based on oxygen (≥approximately 20%).

### BACKGROUND

Inhaled nitric oxide (NO or iNO) is a gaseous medicament commonly used to treat patients suffering from acute pulmonary arterial hypertension, in particular pulmonary vasoconstriction in adults or children, including newborns (PPHN), as described for example in EP-A-560928 or EP-A-1516639.

To implement therapy by inhaled NO, use is made of a gas supply installation, also called an NO administration installation, comprising an NO delivery apparatus and a medical ventilator, that is to say a respiratory assistance apparatus, supplying a patient circuit that generally comprises one or more flexible ducts which are fluidically connected to a respiratory interface, such as a tracheal intubation tube or the like, serving to deliver to the patient to be treated a final gas mixture containing NO.

A gas supply installation of this kind is described, for example, in EP3821929. This type of installation is used in a hospital environment to administer the treatment by NO and thereby to care for patients who need to inhale NO in order to treat their pulmonary arterial hypertension. Installations of this type are also described in EP3233171, EP3410927, EP3410927, EP4209243, EP4241817, EP4241812 and EP4295882.

In such an installation, the NO delivery apparatus makes it possible to inject a gas flow based on NO, typically an NO/nitrogen gas mixture, into the patient circuit, which is also supplied with a respiratory gas flow containing oxygen (at least approximately 20 vol %), such as air or an oxygen/nitrogen mixture (O₂/N₂), supplied by the medical ventilator, so as to obtain a combined flow, also called the “final gas mixture”, comprising NO at the desired dosage, typically less than 50 ppmv of NO, at least approximately 20 vol % of oxygen and nitrogen (N₂), and even some unavoidable impurities.

Means for controlling the flow rate in the apparatus make it possible to control or adjust the flow rate of gas containing NO, e.g. NO/N2 mixture, in order to obtain the desired combined gas mixture, i.e. the final mixture containing NO at the desired dosage.

To be specific, the flow rate of NO-based gas depends in particular on the flow rate of oxygen-based respiratory gas, i.e. air or N₂/O₂ mixture, coming from the medical ventilator. It is therefore necessary to continuously measure the flow rate of oxygen-based respiratory gas using a flow rate sensor arranged in the patient circuit, upstream of the site of injection of the NO-based mixture, and to use these measurements to calculate the flow rate of NO-based gas to be supplied.

However, in practice, fluctuations are observed to a greater or lesser extent in the NO content of the combined gas mixture, these fluctuations being caused by variations in the flow rate of the oxygen-based respiratory gas supplied by the medical ventilator.

As will be appreciated, such variations can be critical for the patient if they are too great and result in an NO content that is (very) different to the desired dosage, in other words if the NO content is excessive or insufficient.

In order to guarantee increased safety for the patient, the user, for example healthcare personnel, can generally set, on the NO delivery apparatus, maximum and minimum values (i.e. upper and lower thresholds) for NO content acceptable in the combined gas mixture.

By ensuring that the composition of the combined gas mixture obtained is monitored, i.e. kept under continuous observation, it is possible to ensure that the proportion of NO therein does not stray too far from the desired dosage. To be specific, in the event that the NO content measured exceeds either the upper or lower threshold, an acoustic alarm and/or a visual alarm may be triggered by the NO delivery apparatus in order to alert the healthcare personnel and allow them to take corrective measures.

However, setting the upper and lower NO alarm thresholds corresponding to the acceptable maximum and minimum NO contents (thresholds) is no easy task because these thresholds depend heavily on the selected dose. Therefore, healthcare personnel may make mistakes when calculating and/or subsequently inputting these thresholds into the NO delivery apparatus, and it is obvious that any error can place the patient in danger.

Furthermore, when the healthcare personnel, for example a doctor, decides to change the dose of NO in order to adjust the patient's treatment, in other words to increase or reduce the dose of NO in the combined gas mixture delivered to the patient, they must also remember to modify the upper and lower NO alarm thresholds that were set for the previous dose and select new ones corresponding to the new dose of NO selected by the doctor.

Here again, in addition to the fact that the doctor may forget to adapt the alarm thresholds to the new dose of NO, mistakes may be made when inputting the thresholds, with the same negative consequences for the patient as mentioned above.

In all cases, alarm thresholds that are incorrectly selected or incorrectly set inevitably lead to the untimely triggering of alarms, which notably results in irritating noise and is tiring for healthcare personnel who have to intervene constantly to deactivate these alarms, thereby distracting them from more critical tasks. This is detrimental to the patient's treatment, in particular their safety.

US2022106189 proposes a system for producing, from NO₂, a gas mixture containing NO that can be administered to a patient, which system includes a gas analyser. Once a dose has been set, the system calculates upper and lower alarm thresholds. The only example given relates to the calculation of thresholds of 7 ppm and 13 ppm, for a dose of NO equal to 10 ppm, in other words a difference of +/−30%. However, nothing is specified regarding the recalculation of the thresholds in the event of subsequent modification of the dose of NO, in particular for doses below 10 ppm.

U.S. Pat. No. 11,833,309 also proposes a device for generating NO, in which the alarm thresholds are calculated or recalculated automatically when a dose of NO is set or changed. This document specifies that either the thresholds can be calculated as percentages around the desired NO value, or use can be made of a pre-established look-up table. However, no information is given regarding the percentage to be applied and the issue of doses below 10 ppm is not addressed.

Moreover, US2013/118486 and US2013/192595 teach an apparatus for administering NO with monitoring of the NO content supplied, in which an alarm is triggered when a difference is determined. Although difference values of between +/−1% and +/−100% are mentioned, it is specified that the difference is preferably at least 25%. The issue of doses below 10 ppm is not addressed therein, in particular as regards recalculation of thresholds following a change in the initial dose.

Therefore, a problem to be solved is that of improving the safety of the patient, in particular being able to prevent or at least minimize the risks mentioned above and/or untimely triggering of the alarm, while ensuring effective treatment for the patient, by proposing an improved NO delivery apparatus, in other words one that is safer from the viewpoint of determining NO alarm thresholds, typically in the event of a change in dose of NO, in particular for low doses of NO, in other words doses below 10 ppmv.

### SUMMARY

One solution concerns an apparatus for delivering, i.e. supplying, a gas containing NO, such as an NO/N₂ gas mixture, comprising means for setting a dose of NO configured to allow a user to set, i.e. fix, select or the like, a first dose of NO (D_{NO1}) of between 1 and 80 ppmv, and operating means with a microprocessor, configured to determine on the basis of said first dose of NO set (D_{NO1}), first upper and lower alarm thresholds corresponding to a first maximum NO content (Tₘₐₓ₁) and to a first minimum NO content (Tₘᵢₙ₁), wherein: Tₘₐₓ₁>D_{NO1}>Tₘᵢₙ₁.

Moreover, the delivery apparatus of the invention further includes means for modifying a dose of NO configured to allow a user to modify or adjust the first dose of NO (D_{NO1}) so as to obtain or fix a second dose of NO (D_{NO2}) that is different to the first dose of NO (D_{NO1}), and the operating means are configured to automatically determine, i.e. calculate, modify, adjust or the like, on the basis of said second dose of NO (D_{NO2}), second upper and lower NO alarm thresholds that are different to said first upper and lower NO alarm thresholds, corresponding to a second maximum NO content (Tₘₐₓ₂) and to a second minimum NO content (Tₘᵢₙ₂), wherein: Tₘₐₓ₂>D_{NO2}>Tₘᵢₙ₂.

Moreover, the operating means are configured so that the second maximum NO content (Tₘₐₓ₂) and the second minimum NO content (Tₘᵢₙ₂) automatically determined are such that:

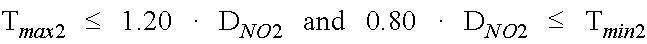

with the additional condition that, when the second dose of NO (D_{NO2}) is below 10 ppmv, said second maximum and minimum NO contents (Tₘₐₓ₂, Tₘᵢₙ₂) are such that: Tₘₐₓ₂−D_{NO2}≥2 ppmv and D_{NO2}−Tₘᵢₙ₂≥2 ppmv.

Preferably, second maximum and minimum NO contents (Tₘₐₓ₂, Tₘᵢₙ₂) are such that: Tₘₐₓ₂−D_{NO2}=2 ppmv and D_{NO2}−Tₘᵢₙ₂=2 ppmv.

In other words, at the start of treatment of a patient, that is to say just before starting the supply of NO to the patient, the operating means of the apparatus automatically determine, i.e. calculate, the values of the first upper and lower NO alarm thresholds, in other words said first maximum and minimum NO contents (Tₘᵢₙ₁, Tₘₐₓ₁), on the basis of the value of the first dose of NO (D_{NO1}) or dosage set by the user, i.e. the doctor or the like, by adding or, conversely, removing from 10% to 20% to or from the value of the dose of NO (D_{NO}) set, preferably around 20% which corresponds to an acceptable tolerance, doing so for a value of a first dose of NO (D_{NO1}) generally between 1 and 80 ppmv.

Then, if the user, typically a doctor, decides to change the dosage, in other words to modify the dose of NO to change it from the first dose of NO (D_{NO1}) to a second dose of NO (D_{NO2}) that is different to the first dose of NO (D_{NO1}), then the apparatus, typically its microprocessor, will automatically calculate, on the basis of this second dose of NO (D_{NO2}), second upper and lower NO alarm thresholds that are different to said first upper and lower NO alarm thresholds, corresponding to second maximum NO (Tₘₐₓ₂) and minimum NO (Tₘᵢₙ₂) contents, and wherein: Tₘₐₓ₂>D_{NO2}>Tₘᵢₙ₂.

The calculation is performed as for the first alarm thresholds, in other words by applying the same tolerance, which is less than or equal to 20%, for example 20%.

The fact that the NO alarm thresholds are recalculated automatically and immediately, in other words instantaneously, by the NO delivery apparatus as soon as the user has set the desired second dose of NO (D_{NO2}), in other words the desired second dosage, is an undeniable advantage in terms of safety because there are no longer any errors in calculating or inputting values into the apparatus or no possibility that the healthcare personnel will forget to recalculate these values.

Furthermore, when the second dose of NO (D_{NO2}) has a low value, in other words when it is below 10 ppmv, the apparatus will automatically calculate, on the basis of this second dose of NO (D_{NO2}), specific second upper and lower NO alarm thresholds which differ by at least 2 ppmv from the new dose of NO (D_{NO2}) which was set or selected by the user, i.e. doctor or the like, preferably a difference of 2 ppmv. This prevents alarms being triggered accidentally, in other words false alarms, and patient safety is thereby improved, as is the effectiveness of the treatment, in particular for paediatric patients.

Depending on the embodiment in question, the delivery apparatus of the invention may comprise one or more of the following features:
the operating means are configured to automatically calculate (i.e. determine), on the basis of the first dose of NO (D_{NO1}) set, a first upper NO alarm threshold corresponding to a first maximum NO content (Tₘₐₓ₁), such that: 1.1. D_{NO1}≤Tₘₐₓ₁≤1.2·D_{NO1}, in other words, such that the first maximum NO content (Tₘₐₓ₁) is greater by 10% to 20% than the first value of a dose of NO (D_{NO1}) set.
the operating means are configured to automatically calculate (i.e. determine), on the basis of the first dose of NO (D_{NO1}) set, a first lower NO alarm threshold corresponding to a first minimum NO content (Tₘᵢₙ₁), such that: 0.8·D_{NO1}≤Tₘᵢₙ₁≤0.9·D_{NO1}, in other words, such that the first minimum NO content (Tₘᵢₙ₁) is less by 10% to 20% than the first value of a dose of NO (D_{NO1}) set.
the operating means are configured to calculate (i.e. determine), on the basis of the first dose of NO (D_{NO1}) set, a first upper NO alarm threshold corresponding to a first maximum NO content (Tₘₐₓ₁), such that:
   Tₘₐₓ₁≤1.20·D_{NO1}, in particular such that: Tₘₐₓ₁=1.20·D_{NO1}.
the operating means are configured to calculate (i.e. determine), on the basis of the first dose of NO (D_{NO1}) set, a first lower NO alarm threshold corresponding to a first minimum NO content (Tₘᵢₙ₁), such that:
   0.80·D_{NO1}≤Tₘᵢₙ₂, in particular such that: 0.80·D_{NO1}=Tₘᵢₙ₁.
according to a preferred embodiment, the operating means are configured to calculate (i.e. determine), on the basis of the first dose of NO (D_{NO1}) set, first upper and lower NO alarm thresholds differing by no more than 20% from the first dose of NO (D_{NO1}), that is, in other words:
   a first upper NO alarm threshold corresponding to a first maximum NO content (Tₘₐₓ₁), such that: Tₘₐₓ₁≤1.20·D_{NO1}, preferably: Tₘₐₓ₁=1.20·D_{NO1}, and
   a first lower NO alarm threshold corresponding to a first minimum NO content (Tₘᵢₙ₁), such that: 0.80·D_{NO1}≤Tₘᵢₙ₁, preferably: 0.80·D_{NO1}=Tₘᵢₙ₁.
advantageously, the operating means are configured to calculate a first maximum NO content (Tₘₐₓ₁) such that: Tₘₐₓ₁−D_{NO1}≥2 ppmv, in other words such that the difference between the first dose of NO (D_{NO1}) set and the first maximum NO content (Tₘₐₓ₁) must be at least 2 ppmv, in particular when the first dose of NO (D_{NO1}) is below 10 ppmv. For example, for a first dose of NO (D_{NO1}) of 10 ppmv, the first maximum NO content (Tₘₐₓ₁) must be set at at least 12 ppmv, i.e. 12 ppmv or above, whereas, for example, for a dose of 5 ppmv, the first maximum NO content (Tₘₐₓ₁) must be set at at least 7 ppmv (i.e. ≥7 ppm).
the operating means are further configured to calculate a first minimum NO content (Tₘᵢₙ₁) such that: D_{NO1}−Tₘᵢₙ₁≥2 ppmv, in other words such that, again, the difference between the first dose of NO (D_{NO1}) set and the first minimum NO content (Tₘᵢₙ₁) must be at least 2 ppmv, in particular when the first dose of NO (D_{NO1}) is below 10 ppmv. For example, for a first dose of NO (D_{NO1}) of 10 ppmv, the first minimum NO content (Tₘᵢₙ₁) must be set at no more than 8 ppmv, i.e. 8 ppmv or below, whereas, for example, for a dose of 5 ppmv, the first minimum NO content (Tₘᵢₙ₁) must be set at no more than 3 ppmv (i.e.≤3 ppm).
it comprises a graphical display means, in other words a display screen or the like.
the graphical display means is configured to display at least said first dose of NO set (D_{NO1}) and/or said first maximum and minimum NO contents (Tₘᵢₙ₁, Tₘₐₓ₁) corresponding to said first upper and lower alarm thresholds.
the graphical display means comprises a touchscreen (i.e. with a touch panel) or any other equivalent display device.
the operating means are configured to manage a display, on the graphical display means, of the first maximum NO content (Tₘₐₓ₁) corresponding to the first upper alarm threshold.
the operating means are configured to manage a display, on the graphical display means, of the first minimum NO content (Tₘᵢₙ₁) corresponding to the first lower alarm threshold.
the first dose of NO (D_{NO1}) is between 1 and 60 ppmv.
the operating means are moreover configured to automatically determine, e.g. calculate, on the basis of the second dose of NO (D_{NO2}) set, a second upper NO alarm threshold corresponding to a second maximum NO content (Tₘₐₓ₂) and a second lower NO alarm threshold corresponding to a second minimum NO content (Tₘᵢₙ₂), said determination being carried out in the same way as for the first upper NO alarm threshold corresponding to a first maximum NO content (Tₘₐₓ₁) and the first lower NO alarm threshold corresponding to a first minimum NO content (Tₘᵢₙ₁).
the operating means are moreover configured to automatically calculate (i.e. determine), on the basis of the second dose of NO (D_{NO2}) set, a second upper NO alarm threshold corresponding to a second maximum NO content (Tₘₐₓ₂), such that: 1.1·D_{NO2}≤Tₘₐₓ₂≤1.2·D_{NO2}, in other words, such that the second maximum NO content (Tₘₐₓ₂) is greater by 10% to 20% than the second value of a dose of NO (D_{NO2}) set.
the operating means are configured to automatically calculate, on the basis of the second dose of NO (D_{NO2}) set, a second lower NO alarm threshold corresponding to a second minimum NO content (Tₘᵢₙ₂), such that: 0.7·D_{NO2}≤Tₘᵢₙ₂≤0.8. D_{NO2}, in other words, such that the second minimum NO content (Tₘᵢₙ₂) is less by 10% to 20% than the second value of a dose of NO (D_{NO2}) set.
the operating means are configured to calculate, on the basis of the second dose of NO (D_{NO2}) set, a second upper NO alarm threshold corresponding to a second maximum NO content (Tₘₐₓ₂), such that: Tₘₐₓ₂≤1.20·D_{NO2}, in particular such that: Tₘₐₓ₂=1.20·D_{NO2}.
the operating means are configured to calculate, on the basis of the second dose of NO (D_{NO2}) set, a second lower NO alarm threshold corresponding to a second minimum NO content (Tₘᵢₙ₂), such that: 0.80·D_{NO2}≤Tₘᵢₙ₂, in particular such that: 0.80·D_{NO2}=Tₘᵢₙ₂.
again, advantageously, the operating means are configured to calculate a second maximum NO content (Tₘₐₓ₂) such that:
   Tₘₐₓ₂−D_{NO2}≥2 ppmv, in other words such that the difference between the second dose of NO (D_{NO2}) set and the second maximum NO content (Tₘₐₓ₂) must be at least 2 ppmv, in particular when the second dose of NO (D_{NO2}) is below or equal to 10 ppmv.
by analogy, the operating means are further configured to calculate a second minimum NO content (Tₘᵢₙ₂) such that: D_{NO2}−Tₘᵢₙ₂≥2 ppmv, in other words such that, again, the difference between the second dose of NO (D_{NO2}) set and the second minimum NO content (Tₘᵢₙ₂) must be at least 2 ppmv, in particular when the second dose of NO (D_{NO2}) is below or equal to 10 ppmv.
the graphical display means is further configured to display at least said second dose of NO (D_{NO2}) and/or said second maximum and minimum NO contents (Tₘᵢₙ₂, Tₘₐₓ₂) corresponding to said second upper and lower NO alarm thresholds that were determined.
the operating means are configured to manage a display, on the graphical display means, of the second maximum NO content (Tₘₐₓ₂) corresponding to the second upper NO alarm threshold and the second minimum NO content (Tₘᵢₙ₂) corresponding to the second lower NO alarm threshold.
the second dose of NO (D_{NO2}) is between 1 and 80 ppmv.
the second dose of NO (D_{NO2}) is between 1 and 75 ppmv, preferably between 1 and 55 ppmv.
the second dose of NO (D_{NO2}) is lower than the first dose of NO (D_{NO1}) or, alternatively, the second dose of NO (D_{NO2}) is higher than the first dose of NO (D_{NO1}).
the difference (in absolute value) between the first dose of NO (D_{NO1}) and the second dose of NO (D_{NO2}) is at least 2 ppmv, preferably at least 3 ppmv, preferably at least 4 ppmv, for example at least 5 ppmv.
the graphical display means is configured to simultaneously display the second dose of NO (D_{NO2}) and the second maximum and minimum NO contents (Tₘᵢₙ₂, Tₘₐₓ₂) corresponding to said second upper and lower NO alarm thresholds that were determined.
the operating means are configured to determine the second minimum and maximum NO contents (Tₘᵢₙ₂, Tₘₐₓ₂) such that, when: D_{NO2}<D_{NO1} then: Tₘₐₓ₂<Tₘₐₓ₁ and Tₘᵢₙ₂<Tₘᵢₙ₁.
the operating means are configured to determine the second minimum and maximum NO contents (Tₘᵢₙ₂, Tₘₐₓ₂) such that, when: D_{NO2}>D_{NO1} then: Tₘₐₓ₂>Tₘₐₓ₁ and Tₘᵢₙ₂>Tₘᵢₙ₁.
it further comprises storage means for storing the first dose of NO (D_{NO1}) and/or the second dose of NO (D_{NO2}).
the storage means make it possible to store the first maximum NO content (Tₘₐₓ₁) and/or the first minimum NO content (Tₘᵢₙ₁).
the storage means make it possible to store the second maximum NO content (Tₘₐₓ₂) and/or the second minimum NO content (Tₘᵢₙ₂).
it comprises an internal gas circuit for conveying a flow of NO-containing gas, in other words gas passages, gas ducts or the like.
the internal gas circuit comprises flow rate control means configured to control the flow of NO-containing gas within said gas circuit.
the operating means are configured to manage (at least) the flow rate control means to control the supply of NO-containing gas as a function of at least said first dose of NO set (D_{NO1}) or, depending on the circumstances, the second dose of NO (D_{NO2}), in other words when the dose of NO has been modified, going from the first dose of NO (D_{NO1}) to the second dose of NO (D_{NO2}).
the internal gas circuit of the apparatus conveying the NO-containing gas, typically an NO/N₂ gas mixture, is fluidically connected to a respiratory circuit conveying a respiratory gas containing oxygen, such as air or an O₂/N₂ gas mixture (>20% O₂), in order to inject the NO-containing gas into it and obtain a combined gas mixture containing NO and oxygen, in other words the final mixture to be administered to the patient.
it comprises NO concentration measurement means for determining the NO content in the combined gas mixture containing NO and oxygen, such as an NO sensor, for example with an electrochemical cell.
the NO concentration measurement means are arranged in such a way as to determine the NO content in the combined gas mixture within the internal gas circuit.
it comprises alarm means, typically an acoustic and/or visual alarm.
the operating means are configured to act on alarm means in order to trigger an alarm, in particular an acoustic and/or visual alarm.
the alarm means can comprise a buzzer-type device, and/or a loudspeaker, or any other device for emitting an acoustic signal that can be heard by the human ear, i.e. by the healthcare personnel.
the alarm means may comprise a luminous device, for example comprising one or more diodes (LEDs) or the like.
the alarm means may comprise the display of an alert message or the like on the graphical display means.
the operating means are configured to trigger an alarm, i.e. to activate alarm means, when the NO content (i.e. an instantaneous content) in the combined gas mixture is higher than or equal to the first or, depending on the circumstances, the second maximum NO content (Tₘₐₓ₁, Tₘₐₓ₂) or lower than or equal to the first or, depending on the circumstances, the second minimum NO content (Tₘᵢₙ₁, Tₘᵢₙ₂).
the operating means determine the NO content (i.e. an instantaneous content) in the combined gas mixture on the basis of measurements supplied by the NO concentration measurement means.
the operating means are configured to manage a display, on the graphical display means, of the NO content in the combined gas mixture that has been determined by the NO concentration measurement means.
the display means is configured to display an alarm message, in the event of an alarm being triggered.
the means for setting a dose and/or the means for modifying a dose of NO are configured to allow the user to modify, set, adjust or the like the dose of NO (D_{NO1}, D_{NO2}) and/or the upper and/or lower NO alarm thresholds (Tₘₐₓ₁, Tₘₐₓ₂, Tₘᵢₙ₁, Tₘᵢₙ₂).
the means for setting a dose and/or the means for modifying a dose of NO comprise one or more keys or the like, in particular virtual keys displayed by the display screen.
it is supplied with a gas containing NO in a given initial proportion, typically an NO/N₂ mixture.
it is supplied with a gas containing an initial proportion of NO of between 100 and 2000 ppmv, preferably between 100 and 1500 ppmv, typically between 200 and 1000 ppmv.
it is supplied with a gas mixture formed of nitrogen and NO.
the means for setting or modifying a dose of NO are configured to allow the user to set, adjust or modify a dose of NO, i.e. NO content setpoint, corresponding to the desired proportion of NO in the combined gas mixture, in other words after mixing of the NO/N₂ mixture with the flow of oxygen-based respiratory gas, i.e. an NO dosage.
the means for setting or modifying a dose of NO form part of an HMI (human-machine interface) or GUI (graphical user interface).
the HMI comprises the graphical display means.
preferably, the means for setting a dose or modifying a dose of NO comprise one or more touch keys that can be operated by the user, displayed on a graphical display means such as a digital screen with a touch panel, in other words virtual keys.
the means for setting a dose and/or the means for modifying a dose of NO comprise a “+” key for incrementing an NO dose value and/or a “−” key for decrementing an NO dose value. Typically, the dose value is incremented or decremented by +/−1 ppmv.
the means for setting a dose and/or the means for modifying a dose of NO further comprise a confirmation key for confirming/validating an NO dose value that has been set, preferably a virtual confirmation key displayed by the display screen.
alternatively, the means for setting or modifying a dose of NO comprise a mechanical selection member, such as one or more buttons or keys operated with the fingers, such as a push button or the like, or a rotary knob or the like, for selecting, choosing, adjusting, fixing, setting, confirming or the like, an NO dose value, such as a starting value or a new value in the event of modification of the dose.
the calculation of the alarm thresholds by the operating means, in particular in the event of modification of the dose of NO to be administered, i.e. the second dose of NO (D_{NO2}), is performed automatically and instantaneously, in other words in real time and in response to the modification of the dose of NO to be administered, typically after confirmation of the new dose by the operator.
the graphical display means is a colour display.
the means for setting or modifying a dose of NO are configured to allow the user to set a dose of NO, i.e. an NO content setpoint.
the NO content setpoints or doses (D_{NO1}, D_{NO2}) are between 1 and 70 ppmv, preferably between 1 and 60 ppmv, advantageously between 1 and 50 ppmv, typically between 5 and 40 ppmv.
the storage means comprise a computer memory, for example a flash memory, RAM or the like.
the flow rate control means are arranged on the internal gas circuit.
the flow rate control means comprise valve means, such as solenoid valves or the like.
the operating means are configured to control the flow rate control means so as to allow or prevent the passage of the flow of NO/N₂ in the internal gas circuit.
the flow rate control means comprise one or more proportional solenoid valves and/or one or more all-or-nothing solenoid valves.
the main flow rate control means comprise a mass flow controller.
the mass flow controller or MFC comprises at least one proportional solenoid valve which is controlled by the operating means, and a main flow rate sensor.
the operating means with a microprocessor comprise a (micro)controller or the like.
the operating means with a microprocessor comprise one or more (micro) processors arranged on one or more electronic boards.
the operating means comprise one or more (micro) processors implementing one or more algorithms, in particular one or more algorithms for operating the flow rate control means, one or more algorithms for processing the respiratory gas flow rate measurements or NO, NO₂ and/or O₂ concentration measurements, etc.
the storage means are arranged on the electronic board.

The invention also relates to an installation for supplying a gas containing NO, comprising the NO delivery apparatus according to the invention supplied with an NO/N₂ gas mixture by an NO/N₂ mixture source, and a medical ventilator configured to supply a flow of respiratory gas containing O₂, typically to a respiratory circuit conveying the flow of respiratory gas leaving the medical ventilator.

Depending on the embodiment in question, the installation of the invention for supplying a gas containing NO may comprise one or more of the following features:
the medical ventilator is configured to supply a flow of respiratory gas containing at least 20 vol % approximately of O₂, typically an NO/N₂ gas mixture or air.
the NO delivery apparatus and the medical ventilator are fluidically connected to the respiratory circuit in order to supply it with gas flows.
a flow rate sensor is arranged in the respiratory circuit between the medical ventilator and the injection device.
an injection device is arranged in the respiratory circuit, downstream of the flow rate sensor.
the injection device is configured to allow injection of the gas containing NO coming from the NO delivery apparatus into the flow of respiratory gas containing O₂ coming from the medical ventilator in order to obtain the combined gas mixture to be supplied to the patient needing same.
the injection device is configured to mix the gas containing NO coming from the NO delivery apparatus with the flow of respiratory gas containing O₂ conveyed through the respiratory circuit, and to obtain a combined gas mixture containing NO and oxygen, or even other compounds such as nitrogen or unavoidable impurities.
the injection device comprises a first gas inlet supplied with a flow of respiratory gas containing O₂, i.e. coming from the medical ventilator.
the injection device also comprises a second gas inlet supplied with gas containing NO coming from the NO delivery apparatus.
the injection device also comprises a gas outlet supplying the combined gas mixture containing NO and oxygen, obtained by mixing, within the injection device, the gas containing NO (e.g. NO/N₂ mixture) with the flow of respiratory gas containing O₂ (e.g. air or O₂/N₂ mixture).
the medical ventilator delivers air or an oxygen/nitrogen mixture, i.e. as a respiratory gas containing at least 20 vol % approximately of oxygen, preferably at least 21 vol % approximately of oxygen.
the medical ventilator comprises a motorized blower (i.e. turbine, compressor or similar) delivering the respiratory gas, typically air or an oxygen/nitrogen mixture or, according to another embodiment, an internal gas circuit comprising one or more proportional valves for conveying the gas and controlling its supply, in particular its flow rate. Such a ventilator is generally supplied with respiratory gas via one or more wall outlets supplied with gas from a network of pipes in a hospital establishment or building, typically with air or an oxygen/nitrogen mixture.
the medical ventilator comprises control means or a control device, such as one or more electronic control boards. Preferably, the control means of the medical ventilator operate or control the motorized blower or, depending on the circumstances, the proportional valves of the medical ventilator.
the medical ventilator is of HFO type or comprises an HFO function, that is to say it is able to produce high-frequency oscillations.
the source(s) of NO supplying the NO delivery apparatus contain(s) an NO/N₂ gas mixture containing between 100 and 2000 ppmv of NO, the remainder being nitrogen (N₂), preferably between 100 and 1000 ppmv of NO, at a pressure of between 10 and 250 bar abs, typically at over 100 bar abs (before start of withdrawal).
the source of NO is or comprises one or more gas cylinders with a content of between 0.5 and 50 L (equivalent in water).
the gas cylinder(s) comprises a cylindrical body made of steel or aluminium alloy and is equipped with a simple valve (without regulator) or a valve with an integrated regulator or IRV, preferably an IRV protected by a protective cap, for example made of metal or polymer.
the respiratory circuit (i.e. patient circuit) comprises an inhalation branch and an exhalation branch, typically flexible ducts forming the inhalation branch and the exhalation branch, for example polymer hoses.
the inhalation branch and the exhalation branch, e.g. flexible ducts, are connected to a joining piece, such as a Y-piece.
the inhalation branch and/or the exhalation branch are fluidically connected to a patient respiratory interface, preferably via the joining piece.
the patient respiratory interface comprises a tracheal intubation tube or a breathing mask or the like.
the respiratory circuit, in particular the inhalation branch, may comprise a gas humidifier.
the gas humidifier is arranged downstream of the injection device, for example an NO injection module, so as to be able to humidify the gas before it is administered to the patient by inhalation.
the ventilator and the NO delivery apparatus are supplied with power by one or more electric current sources, typically the mains supply (110/220 V) and/or one or more rechargeable batteries.

According to another aspect, the invention also relates to a method for therapeutic treatment of a person, i.e. a human patient (i.e. adult, child, adolescent or neonate), suffering from pulmonary hypertension and/or hypoxia, which cause pulmonary vasoconstriction or similar, said method comprising administration by inhalation, to the person requiring it, of a gas mixture comprising from 1 to 80 ppmv of NO, typically less than 40 ppmv, and approximately at least 20 vol % of oxygen, preferably approximately at least 21 vol % of oxygen, by means of a gas supply installation, as described above according to the invention, comprising an NO delivery apparatus according to the invention for delivering NO, so as to treat (at least partially) said pulmonary hypertension and/or said hypoxia, which can be caused by one or more pulmonary diseases or disorders typically such as PPHN (persistent pulmonary hypertension of the newborn) or ARDS (acute respiratory distress syndrome), or can be caused by heart surgery with the patient being placed on extracorporeal blood circulation (ECC).

### DEFINITIONS

In general, in the context of the invention:
“ppmv” means parts per million by volume.
“vol %” means percentage by volume.
“NO” denotes nitric oxide.
“NO₂” denotes nitrogen dioxide.
“N₂” denotes nitrogen.
“O₂” denotes oxygen.
the terms “concentration”, “quantity”, “proportion”, “dose” and “content” are considered to be equivalent and interchangeable.
the terms “means of/to/for” are considered to be wholly equivalent to and interchangeable with the terms “device of/to/for”, for example the term “operating means” may be replaced by “operating device”, the term “valve means” may be replaced by “valve device”, the term “storage means” may be replaced by “storage device”, etc.
“flow rate measurement” means a flow rate value (e.g. a numerical value) or a signal representative of such a flow rate value, which reflects or corresponds to a gas flow rate measured by a flow rate sensor, such as a mass flow sensor.
“pressure measurement” means a pressure value (e.g. a numerical value) or a signal representative of such a pressure value, which reflects or corresponds to the gaseous pressure measured by a pressure sensor.
“concentration measurement” means a value for the content of a given gaseous compound, such as NO, NO₂ or O₂, that is to say a numerical value or a signal representative of such a content value, which reflects or corresponds to the proportion (i.e. quantity) of the gaseous compound considered in a given gas mixture, measured by concentration measurement means, such as electrochemical cell sensors.

### BRIEF DESCRIPTION OF VIEWS OF DRAWINGS

A clearer understanding of the invention will now be obtained from the following detailed description, given as a non-limiting example, with reference to the appended figures, in which:

FIG. 1 shows schematically an embodiment of a gas administration installation comprising an NO delivery apparatus according to the invention.

FIG. 2 shows schematically an embodiment of the internal architecture of the NO delivery apparatus according to the invention, like that of the installation of FIG. 1.

FIG. 3 shows schematically an embodiment of the display of various pieces of information on the display means of an NO delivery apparatus according to the invention, like that of FIG. 2, in particular a first dose of NO that has been set.

FIG. 4 shows schematically a modification of the dose of NO by the user.

FIG. 5 is similar to FIG. 3 but shows schematically the display obtained after modification of the dose of NO by the user.

### DETAILED DESCRIPTION

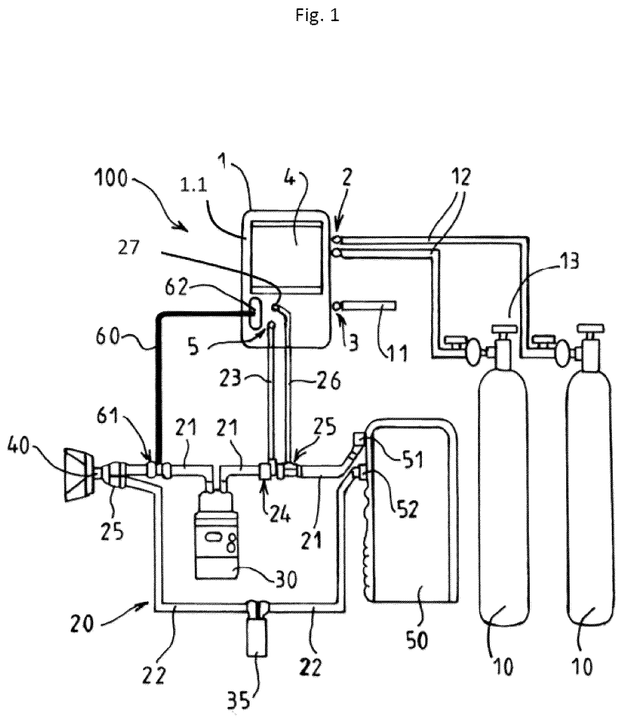

FIG. 1 shows schematically an embodiment of a gas administration installation 100 according to the invention, comprising an NO delivery apparatus 1 according to the invention for supplying a gas mixture based on nitric oxide (NO), typically an NO/N₂ gas mixture, and a medical ventilator 50 which supplies a gas containing at least 20 vol % of oxygen, typically air, an O₂/N₂ gas mixture, or the like.

In this case, the installation 100 comprises two pressurized gas cylinders 10, each containing an NO-based gas mixture, namely an NO/N₂ gas mixture containing in this case between 100 and 1000 ppmv of NO (remainder N₂), for example 450 or 800 ppmv of NO (remainder N₂), or any other suitable concentration, which feed an NO/N₂ mixture to the device or apparatus 1 for delivering or supplying NO, making it possible to monitor and control the supply of the NO/N₂ gas mixture.

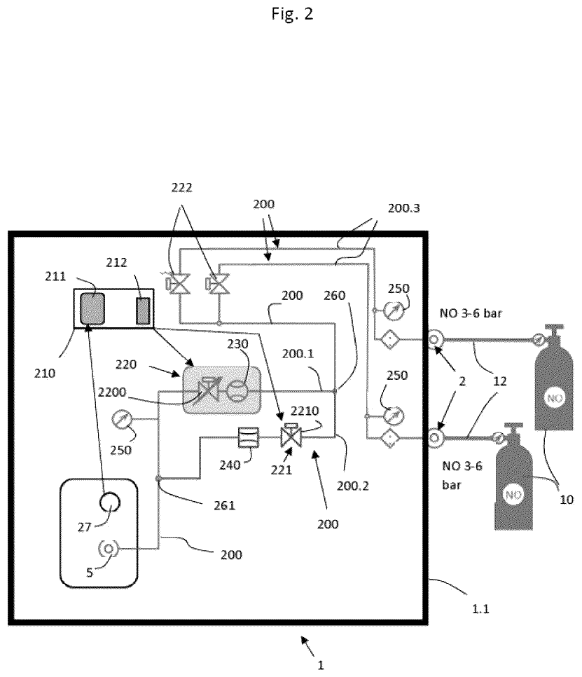

The gas cylinders 10 are fluidically connected to the NO supply apparatus 1 via gas feed lines 12, such as flexible pipes or hoses or the like, which may be equipped with devices for regulating and/or monitoring the gas pressure, such as a gas pressure-relieving valve 13, pressure gauges, etc. The gas feed lines 12 are connected to one or more gas inlets 2 of the NO delivery apparatus 1, which supply an internal gas circuit 200, as shown schematically in FIG. 2, used to convey the gas within the NO supply apparatus 1, i.e. in the outer casing or shell 1.1 of the NO delivery apparatus 1 according to the invention.

In the embodiment of FIG. 2, the internal gas circuit 200 is connected to two gas inlets 2 arranged in parallel and each feeding a dedicated inlet section 200.3 of the internal gas circuit 200. Control valves 222 or the like control the passage of the flow of NO/N₂ in these inlet sections 200.3.

The NO delivery apparatus 1 further comprises an oxygen inlet 3, which is connected fluidically, via an oxygen feed line 11, such as a flexible pipe or the like, to a source of oxygen (not shown), for example a pressurized oxygen cylinder or a hospital network, in other words an oxygen supply pipe which is provided in a hospital building. This makes it possible to feed the internal gas circuit 200 with oxygen when necessary.

The medical ventilator 50, i.e. a respiratory assistance apparatus, supplies a flow of oxygen-based respiratory gas, i.e. containing approximately at least 20 vol % of oxygen, preferably approximately at least 21 vol % of oxygen, such as air or an oxygen/nitrogen (N₂/O₂) mixture.

The medical ventilator 50 and the NO supply apparatus 1 of the installation 100 are in fluidic communication with a respiratory circuit 20, also called the patient circuit, in particular with a gas feed line or inhalation branch 21 of the respiratory circuit 20, which serves to convey the gas flow to the respiratory interface 40 supplying the therapeutic gas flow to the patient, that is to say a combined gas mixture, i.e. final mixture, containing the desired dosage of NO. This combined gas mixture is obtained by mixing the oxygen-based flow (e.g. air or O₂/N₂ mixture) coming from the medical ventilator 50 and the NO-containing flow, i.e. the NO/N₂ gas mixture, delivered by the NO delivery apparatus 1.

For this purpose, the NO delivery apparatus 1 supplies or injects the NO/N₂mixture into the respiratory circuit 20 conveying the oxygen-based flow, via an injection line or pipe 23, which fluidically connects the internal gas circuit 200 of the NO supply apparatus 1 to an injection device 24 arranged on the gas feed line 21.

The injection device 24 is configured to mix the NO-containing gas coming from the NO delivery apparatus 1 with the flow of O₂-containing respiratory gas coming from the ventilator 50 and conveyed by the inhalation branch 21 of the respiratory circuit 20, and to obtain a combined gas mixture containing NO and oxygen, i.e. the final gas mixture administered to the patient.

More specifically, the injection device 24 comprises a first gas inlet supplied with a flow of O₂-containing respiratory gas from the medical ventilator 50, a second gas inlet supplied with NO-containing gas, i.e. gas coming from the NO delivery apparatus 1, and a gas outlet supplying the combined gas mixture containing NO and oxygen, obtained by mixing, within the injection device 24, the NO-containing gas with the flow of O₂-containing respiratory gas.

In other words, the flow of NO/N₂ fed by the injection line 23 is then mixed (by virtue of the injection device 24) with the flow of gas based on oxygen (>20% O₂), e.g. air or an oxygen/nitrogen mixture, delivered by the medical ventilator 50 and conveyed by the inhalation branch 21 of the patient circuit 20, so as to obtain a final mixture, i.e. the combined mixture, which is to be administered to the patient and contains essentially NO at the desired dosage, nitrogen (N₂) and oxygen (O₂), and possibly unavoidable impurities (e.g. argon, CO₂, NO₂, etc.), i.e. a final NO/N₂/O₂ gas mixture.

The inhalation branch 21 of the circuit 20 further comprises a gas humidifier 30 arranged downstream of the injection device 24. This makes it possible to humidify the final gas flow, i.e. the combined NO/N₂/O₂ gas mixture, before it is administered by inhalation to the patient to be treated, by means of a respiratory interface 40, such as a tracheal intubation tube, a breathing mask or the like.

A line for recovering the gases exhaled by the patient forms an exhalation branch 22 of the patient circuit 20. It is fluidically connected to the inhalation branch 21 via a connection piece 25, such as a Y-piece.

At its upstream end, the inhalation branch 21 is fluidically connected to an outlet port 51 of the medical ventilator 50, such as a connector, coupling or the like, so as to recover and convey the oxygen-based gas, typically air or N₂/O₂ mixture, supplied by the medical ventilator 50, while the exhalation branch 22 conveying the exhaled gases is fluidically connected to an inlet port 52 of the medical ventilator 50, such as a connector, coupling or the like, so as to return to the medical ventilator 50 all or part of the flow of the gases exhaled by the patient. The exhalation branch 22 may comprise one or more optional components, for example a CO₂ removal device 35, i.e. a CO₂ trap, such as a hot container or the like, making it possible to remove the CO₂ present in the gases exhaled by the patient, or a filter or the like.

Furthermore, a flow rate sensor 25, for example of hot wire or pressure differential type, is arranged on the respiratory circuit 20, in particular on the inhalation branch 21, between the ventilator 50 and the injection device 24. The flow rate sensor 25 is connected at a connection port to the sensor 27 of the NO delivery apparatus 1 via one or more flow rate measurement lines 26, which are connected at said connection port to the sensor 27. It is used to measure the flow rate of gas delivered by the ventilator 50, such as air or N₂/O₂, circulating in the inhalation branch 21, upstream of the injection device.

These flow rate measurements taken by the flow rate sensor 25 make it possible to control or regulate more efficiently the delivery of the flow of NO (i.e. N₂/O₂) by the NO delivery apparatus 1, in particular the flow rate of NO, since the flow rate measurements taken by the flow rate sensor 25 are returned, via the flow rate measurement line 26 (i.e. electric cables or the like) and the port for connection to the sensor 27, to (micro) processor operating means 210 of the NO delivery apparatus 1, typically a controller, which process these flow rate measurements as explained hereinafter and illustrated in FIG. 2. The port for connection to the sensor 27 is connected electrically to the operating means 210 via one or more electrical connections, for example electric cables or the like.

The NO supply apparatus 1 comprises a rigid casing 1.1, which for example is made of polymer, comprising the internal gas circuit 200 in FIG. 2, typically gas lines, passages or ducts or the like, which fluidically connect the gas inlet (or inlets) 2 of the NO supply apparatus 1 to the injection line 23 so as to convey the flow of gas based on NO between them.

In the embodiment shown schematically in FIG. 2, a portion of the internal gas circuit 200 comprises two gas sections arranged in parallel, i.e. a main section 200.1 and a secondary section 200.2, referred to as the back-up section. The main section 200.1 and the secondary section 200.2 are fluidically connected to one another and to the rest of the gas circuit 200 at upstream 260 and downstream 261 connection sites which are situated respectively upstream and downstream of main and secondary flow rate control means 220, 221.

During normal operation of the apparatus 1, the flow of NO/N₂ passes through the main section 200.1, whereas in the event of a malfunction rendering the main flow rate control means 220 non-operational, the flow of NO/N₂ is diverted and then passes through the back-up section 200.2.

Of course, according to another embodiment (not shown), the internal gas circuit 200 could be configured differently, for example it could comprise a single gas line instead of the two sections 200.1, 200.2. However, in this embodiment, malfunctioning of the main flow rate control means 220 could not be managed, and the apparatus 1 would then become non-functional.

In general, the main and secondary flow rate control means 220, 221, such as main and secondary valve means 2200, 2210, shown schematically in FIG. 2, i.e. one or more valve devices, for example one or more proportional solenoid valves controlled by the operating means 210, are arranged on the internal gas circuit 200, in particular on the main 200.1 and secondary 200.2 sections, and serve to control or adjust the gas flow circulating therein in the direction of the injection line 23, i.e. towards the injection device 24, whether in normal operating mode or in back-up mode.

Preferably, the main section 200.1 comprises a proportional solenoid valve 220 and an additional flow rate sensor 230, typically a mass flow controller or MFC, whereas the secondary section 200.2 comprises one or more solenoid valves of all-or-nothing (AON) type 221, preferably controlled in pulse mode.

Preferably, the main and secondary flow rate control means 220, 221 of the NO supply apparatus 1 are commanded, i.e. controlled, by the operating means 210, i.e. one or more operating devices or (micro) controllers, arranged in the housing 1.1 of the NO supply apparatus 1.

Typically, the operating means 210 comprise one or more electronic boards comprising one (or a plurality of) microprocessor(s) 211 implementing one or more algorithms. The operating means 210 make it possible in particular to adjust or control the flow rate of NO-based gas by operating all or some of the valve means 2200, 2210, typically to open or close one or more (solenoid) valves, so as to obtain a flow of NO-based gas, typically to permit or stop the flow of gas.

Naturally, the operating means 210 also make it possible to perform calculations, and/or to control or command all the electromechanical elements of the apparatus 1, such as sensors, displays, etc.

As explained hereinafter, the operating means 210 can determine the flow rate of NO to be supplied in order to obtain the NO content required in the combined mixture, i.e. the desired NO dosage, in particular on the basis of an NO content setpoint fixed and/or set by the user, i.e. a dose of NO (D_{NO}) set by the user, namely a first dose of NO (D_{NO1}) or subsequently a second dose of NO (D_{NO2}) as explained below, of the composition of the NO/N₂ gas mixture, in particular the NO content in this NO/N₂ gas mixture, which may be stored by the apparatus 1, and of one or more flow rate measurements taken by the flow rate sensor 25 arranged on the inhalation branch 21, and connected by a flow rate measurement line 26 to the NO supply device 1, in particular to the control means 210, via the port for connection to the sensor 27.

The internal gas circuit 200 of the NO supply apparatus 1 may also comprise other elements or components, in particular one or more pressure sensors 250, one or more additional flow rate sensors or flow meters, and/or calibrated-orifice devices 240 or the like. These other elements may be arranged upstream and/or downstream of the flow rate control means 220, 221, i.e. valve means; for example, it is possible to use an additional flow rate sensor in order to determine the flow rate of NO-based gas circulating in all or part of the internal gas circuit 200, in particular in order to ensure that it complies with the desired flow rate.

As can be seen in FIG. 2, the main section 200.1 comprises an additional flow rate sensor 230 arranged upstream of the flow rate control means 220, such as valve means 2200, for example one or more solenoid valves, preferably a proportional solenoid valve, controlling the passage of gas in the main section 200.1. This assembly forms a mass flow controller (MFC).

Furthermore, the secondary section 200.2 comprises a calibrated-orifice device 240 arranged downstream of secondary flow rate control means 221, such as secondary valve means 2210, preferably one or more solenoid valves controlling the flow rate of passage of gas in the secondary section 200.2.

Advantageously, the solenoid valve of the secondary flow rate control means 221 is of all-or-nothing (AON) type, i.e. able to adopt 2 “stable” positions, namely an open position which allows the gas flow to pass, and a closed position which prevents any gas flow from circulating.

Furthermore, the flow meter or additional flow rate sensor 230 of the MFC may be of the pressure differential or mass type, or another type, and interacts with the control means 210 in order to provide them with flow rate measurements of the NO/N₂ flow.

Usually, the NO supply apparatus 1 also comprises a graphical user interface (GUI) comprising a graphical display screen 4, preferably a touch screen, i.e. with a touch panel, serving to display various pieces of information or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

However, according to another embodiment, the apparatus 1 may also comprise one or more mechanical, i.e. non-virtual, selection members, such as one or more selection buttons or keys that may be operated via pressure exerted by the fingers of the user, one or more rotary selection knobs or the like. The selections or choices made via this or these mechanical selection members may be displayed on the graphical display means 4 of the GUI.

In particular, the first dose of NO (D_{NO1}) and the second dose of NO (D_{NO2}) may be set or selected via one or more virtual selection keys and/or keypads or windows displayed by the graphical display means 4, particularly when this has a touch panel.

The electrical power for the NO supply apparatus 1, in particular for the components requiring electrical current in order to operate, such as the operating means 210, the graphical display means 4, etc., is provided conventionally by an electrical current source and/or electrical power supply means (not shown), for example a connection to the mains current (110/220 V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

In addition, the installation 100 also comprises a gas sampling line 60 which fluidically connects the inhalation branch 21 to the NO supply apparatus 1.

The gas sampling line 60 serves to monitor, i.e. keep under observation, the composition of the combined gas mixture containing NO, O₂, etc., in order to ensure that the contents of NO, O₂ and also toxic NO₂ species which can form are in conformity, in particular to ensure that the proportion of NO is well within the fixed upper and lower NO alarm thresholds, i.e. between acceptable maximum and minimum NO contents. If this is not the case, the operating means of the apparatus 1 trigger an acoustic alarm and/or a visual alarm in order to alert the healthcare personnel, by activating (i.e. operating/commanding) the alarm means.

The gas sampling line 60, also called the monitoring line, is fluidically connected, at a connection site 61 in FIG. 1, to the gas feed line 21, between the humidifier 30 and the joining piece 25, i.e. the Y-piece, typically in immediate proximity to the joining piece 25, and also to an inlet port 62 of the NO supply device 1, for example a port 62 carried by a connector, coupling or the like, allowing the connection of the gas sampling line 60, such as a flexible pipe or the like.

The gas sampling line 60 makes it possible to take gas samples and convey them to the NO supply device 1 where they are analysed in an internal gas analyser (not shown), that is to say within a calibration line comprising at least one sensor, in particular one or more electrochemical cells, connected electrically to the operating means, in order to verify their conformity.

As stated above, it should be verified that the composition of the final gas, i.e. the combined gas mixture, conforms with that of the desired NO/N₂/O₂ gas mixture to be administered to the patient, in particular in order to ensure that it does not contain excessive amounts of toxic NO₂ species, that its oxygen content is not hypoxic, and that its NO content corresponds to the desired dosage (neither too high nor too low), i.e. the dose of NO to be administered by inhalation which is selected by the healthcare personnel, i.e. a doctor or the like, in other words the first dose of NO (D_{NO1}) or the second dose of NO (D_{NO2}) in the event of modification of the first dose of NO (D_{NO1}).

This verification of conformity is conventionally carried out by dedicated NO, NO₂ and O₂ concentration measurement means, typically NO₂, NO and O₂ sensors, for example electrochemical cells or the like, which must be calibrated periodically, for example every week.

The operating means 210 of the apparatus 1 are additionally configured to recover and process, i.e. analyse, the signals coming from the various NO, NO₂ and O₂ concentration measurement means, for example sensors, of the gas analyser, which is arranged in the apparatus 1, and to act in response to these signals, in particular to carry out calibration of the sensors, trigger acoustic and/or visual alarms, etc.

In general, the operating means 210, based on a (micro) processor 211, such as a controller, determine the setpoint flow rate for gas containing NO to be supplied to the injection device 24 and operate the flow rate control means 220, 221, for example proportional solenoid valves 220 and/or AON solenoid valves 221, to supply the gas containing NO at the setpoint flow rate. This determination of the setpoint flow rate of NO is carried out on the basis of a respiratory gas flow rate measurement, i.e. a flow rate signal or value, taken and supplied by the flow rate sensor 25 to the operating means 210, of an NO content setpoint corresponding to the final proportion of NO desired in the combined gaseous mixture, typically fixed by the user, i.e. healthcare personnel, and of the initial proportion of NO in the gas containing NO fed to the NO delivery apparatus 1, i.e. the quantity of NO present in the NO/N₂ mixture coming from the gas cylinders 10, typically between 200 and 1000 ppmv, for example 450 or 800 ppmv.

Preferably, the NO content in the NO/N₂ gas mixture fed to the apparatus 1 may be stored by the storage means 212 of the apparatus 1.

Moreover, the NO content in the NO/N₂ gas mixture fed to the apparatus 1 may or may not be adjustable, that is to say stored once and for all or able to be set by the user, within a dedicated menu or the like of the apparatus 1, for example by once again using the setting means, such as one or more keys, rotary knobs, cursors or the like.

The desired first dose of NO (D_{NO1}), which is typically between 1 and 80 ppmv, in other words the NO setpoint value at the start of treatment, must be entered by the user, for example via the HMI, using means for setting a dose or the like, such as one or more keys, rotary knobs, cursors or similar, preferably via one or more virtual keys displayed on the display screen 4.

As explained above, in order to be able to detect and warn the user, in other words the healthcare personnel, in the event that the concentration of NO is excessive or, conversely, insufficient in the combined gas mixture supplied to the patient, it is essential to set the upper and lower NO alarm thresholds, in other words (at least) an upper threshold and a lower threshold, corresponding to the acceptable maximum and minimum NO contents, bearing in mind the fluctuations in NO concentration that may occur as a result of possible variations notably of the flow rate of oxygen-based respiratory gas coming from the medical ventilator 50.

Therefore, just before treatment commences, for example when starting up the apparatus 1, it is necessary to define first upper and lower alarm thresholds corresponding to a first maximum NO content (Tₘₐₓ₁) and a first minimum NO content (Tₘᵢₙ₁), wherein: Tₘₐₓ₁>D_{NO1}>Tₘᵢₙ₁.

These upper and lower NO alarm thresholds (i.e. Tₘₐₓ₁, Tₘᵢₙ₁) are set automatically, via the operating means 210, when the first dose of NO (D_{NO1}) desired by the user, i.e. a doctor for example, is set, thus avoiding errors in calculation and moreover errors linked to inputting these NO alarm thresholds into the NO delivery apparatus. This improves patient safety.

More specifically, to this end, the operating means 210 automatically calculate or determine, on the basis of the first dose of NO set (D_{NO1}), selected by the doctor or the like, the first upper and lower alarm thresholds corresponding to a first maximum NO content (Tₘₐₓ₁) and to a first minimum NO content (Tₘᵢₙ₁), wherein: Tₘₐₓ₁>D_{NO1}>Tₘᵢₙ₁.

Once the desired first dose of NO (D_{NO1}), i.e. desired dosage, typically between 1 and 80 ppmv, preferably lower than or equal to 60 ppmv, more preferably lower than or equal to 40 ppmv, for example around 20 ppmv, has been set by the user using the means for setting a dose of NO, such as one or more virtual keys displayed by the graphical display means 4, the chosen value, corresponding to the first dose of NO (D_{NO1}) set, is used by the operating means 210 to automatically calculate or determine first upper and lower NO alarm thresholds as follows:
the first upper NO alarm threshold corresponds to a first maximum NO content (Tₘₐₓ₁), such that: 1.1·D_{NO1}≤Tₘₐₓ₁≤1.2·D_{NO1}, for example: Tₘₐₓ₁=1.2·D_{NO1}, and
the first lower NO alarm threshold corresponds to a first minimum NO content (Tₘᵢₙ₁), such that: 0.7·D_{NO1}≤Tₘᵢₙ₁≤0.80·D_{NO1}≤Tₘᵢₙ₁, for example: Tₘᵢₙ₁=0.8·D_{NO1}.

In other words, before or during commencement of treatment, the operating means 210 of the apparatus 1 automatically determine the first values of the upper and lower NO alarm thresholds (Tₘᵢₙ₁, Tₘₐₓ₁), by adding or, conversely, subtracting from 10% to 20% (i.e. a tolerance), preferably 20%, to or from the value of the first dose of NO (D_{NO1}) set, as a function of the acceptable tolerance value (%), in other words the fluctuations in NO content in the combined gas mixture that can be tolerated during patient treatment.

The tolerance value (%) maybe non-modifiable, for example factory-set and stored in the apparatus, or, according to another embodiment, may be modifiable by the user, for example within a settings menu of the apparatus 1. The percentage (%) tolerance may depend on various factors, for example with regard to a particular treatment protocol. Advantageously, the tolerance is fixed at 20%.

The tolerance value (%) and/or the calculation formulas including this tolerance value (%) are stored within the apparatus 1, for example in storage means 212. For example, a tolerance of 20% which is applied to the calculation of all NO alarm thresholds.

Once they have been determined, the first maximum NO content (Tₘₐₓ₁) corresponding to the first upper NO alarm threshold and the first minimum NO content (Tₘᵢₙ₁) corresponding to the first lower NO alarm threshold are preferably stored, i.e. recorded, by the storage means 212.

For example, for a first dose of NO (D_{NO1}) set at 20 ppmv by the doctor or the like, the delivery apparatus 1 of the invention, typically its operating means 210, automatically calculates first upper and lower NO alarm thresholds of between 14 and 18 ppmv for the first minimum NO content (Tₘᵢₙ₁) and between 22 and 26 ppmv for the first maximum NO content (Tₘₐₓ₁).

If the percentage (%) tolerance has been fixed at 20% in the settings, for example set in the factory, the above calculation formulas are therefore summed up as follows:
Tₘₐₓ₁=1.20·D_{NO1}, for the first upper NO alarm threshold, i.e. 20% above the first dose of NO (D_{NO1}) set, and
Tₘᵢₙ₁=0.80·D_{NO1} for the first lower NO alarm threshold, i.e. 20% below the first dose of NO (D_{NO1}) set.

Therefore, with the first dose of NO (D_{NO1}) set at 20 ppmv, the apparatus 1 can then automatically calculate first upper and lower NO alarm thresholds at 16 ppmv for the first minimum NO content (Tₘᵢₙ₁) and at 24 ppmv for the first maximum NO content (Tₘₐₓ₁), in other words first upper and lower thresholds at 20 ppmv+/−20% (i.e. +/−4 ppmv).

In all cases, in particular for doses of NO (D_{NO}) set at below 10 ppmv, the operating means 210 are configured to calculate and/or fix first maximum and minimum NO contents (Tₘₐₓ₁, Tₘᵢₙ₁) that preferably differ by at least 2 ppmv from the first dose of NO (D_{NO1}) set, in other words such that: Tₘₐₓ₁−D_{NO1}≥2 ppmv and D_{NO1}−Tₘᵢₙ₁≥2 ppmv.

For example, for a first dose of NO (D_{NO}) set at 5 ppmv and a tolerance of 20%, the operating means 210 are configured to calculate and/or fix first maximum and minimum NO contents (Tₘₐₓ₁, Tₘᵢₙ₁) of 3 ppmv for the first minimum NO content (Tₘᵢₙ₁) and 7 ppmv for the first maximum NO content (Tₘₐₓ₁), in other words +/−2 ppmv relative to the dose of NO (D_{NO}) of 5 ppmv, whereas in theory these should be 4 ppmv and 6 ppmv, respectively, in other words +/−20% relative to the dose of NO (D_{NO}) of 5 ppmv.

To be specific, such a difference of at least 2 ppmv relative to the dose of NO (D_{NO}) set makes it possible to prevent alarms being triggered too frequently while ensuring good patient safety.

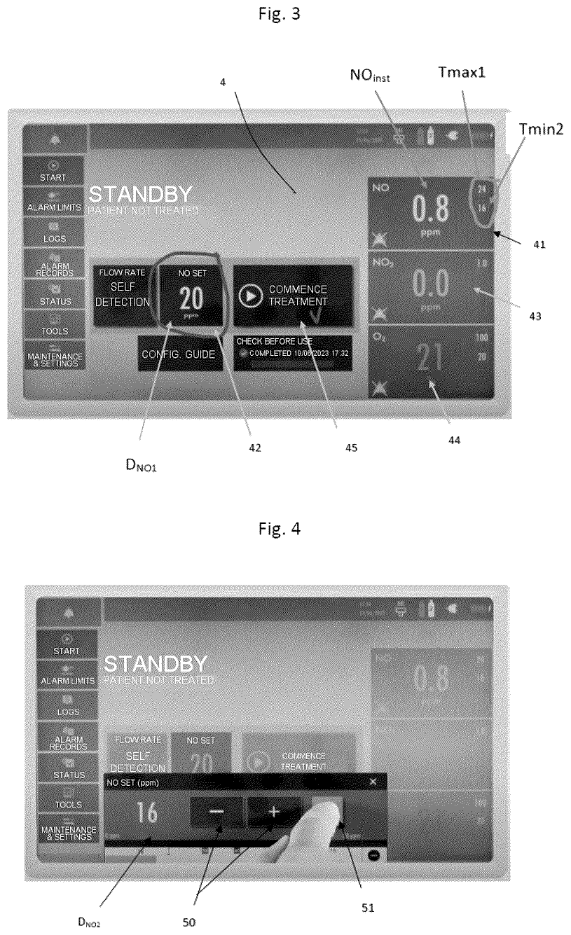

Once the first upper and lower NO alarm thresholds have been determined, they are preferably displayed by the graphical display means 4, as shown in FIG. 3, which schematically shows the display, in a first dedicated window 41, of the first maximum NO content (Tₘₐₓ₁) corresponding to the first upper NO alarm threshold, in this case 24 ppmv, and of the first minimum NO content (Tₘᵢₙ₁) corresponding to the first lower NO alarm threshold, in this case 16 ppmv.

As shown in this figure, the instantaneous NO content value (NOᵢₙₛₜ), in this case 0.8 ppmv, which is measured by the NO concentration measurement means, for example an NO sensor, is also displayed in the first window 41. In this case, as treatment has not yet commenced (the following information is displayed: ATTENTE—Patient Non-Traité (STANDBY—Patient Not Treated)), the value of NO in the flow sent to the patient is almost zero (0.8 ppmv) because the flow of respiratory gas coming from the medical ventilator 50 has not yet been mixed with the flow of NO/N₂ mixture coming from the apparatus 1. Therefore, the NO concentration measurement means only determine a negligible quantity of NO in the flow of air, in other words in the form of traces or unavoidable impurities.

FIG. 3 thus shows the display means 4 after the first dose of NO (D_{NO}) has been fixed by the user and after the first alarm thresholds (Tₘₐₓ₁, Tₘᵢₙ₁) have been calculated, but just before the start of patient treatment, which may then commence after the user has pressed the virtual start-up key 45 displayed on the graphical display means 4. Moreover, as can also be seen, the first dose of NO (D_{NO1}) set, in other words the dosage, is also displayed in a second window 42, in this case 20 ppmv.

The NO concentration measurement means supply the NO measurements (i.e. value or signal) to the operating means 210, which process them so as to trigger alarms if needed and/or manage their display on the display means 4, in order to ensure effective monitoring of the operation of the apparatus 1 and/or of the installation 100.

Furthermore, the display means 4 also displays the concentrations of NO₂ and O₂ measured by the NO₂ and O₂ concentration measurement means, typically NO₂ and O₂ sensors, for example electrochemical cells or the like, and processed by the operating means 210, which also manage their display on the display means 4, in this case in third and fourth windows 43, 44.

As can be seen, the NO₂ content measured and displayed in the third window 43 is in this case equal to 0 ppmv, which is normal since treatment has not yet commenced, and therefore there can have been no oxidation of NO molecules to form toxic NO₂ species.

Conversely, it can be seen that the supply of the flow of respiratory gas containing oxygen, for example air or a gas mixture made up of 21% O₂ and the remainder N₂ for example, coming from the ventilator 50, has started since the O₂ concentration measurement means have detected an oxygen content of 21 vol % approximately in the gas flow present in the respiratory circuit 20. This oxygen content is displayed in the fourth window 44.

Once the first dose of NO (D_{NO1}) has been set by the user and the first upper and lower alarm thresholds corresponding to the first maximum NO content (Tₘₐₓ₁) and minimum NO content (Tₘᵢₙ₁) have been calculated by the apparatus 1, for example 16 and 24 ppmv in FIG. 3 for a dose of 20 ppmv initially fixed or selected by the user, the treatment of a patient can be started by the user, i.e. the healthcare personnel.

The NO is then distributed by the apparatus 1 at a given flow rate making it possible to obtain the desired NO dosage in the combined gas mixture, namely a dosage corresponding to the first dose of NO (D_{NO1}) set by the user. This is done by operating all or some of the flow rate control means 220, 221, as explained above.

However, if the doctor or similar notices that the dose of NO supplied is not right for the patient, in other words that it is too high or too low, then they can modify this first dose of NO (D_{NO1}), via means for modifying a dose of NO, i.e. as previously, one or more virtual keys for example, so as to adopt a second dose of NO (D_{NO2}) different to the first dose of NO (D_{NO1}).

For example, if the first dose of NO (D_{NO1}) that was set, in other words the starting dosage, was 20 ppmv, as shown in FIG. 3, and if the doctor thinks that this is too high for the patient in question, they may decide to reduce it to 16 ppmv for example (or conversely, to increase it if it is too low).

In this case, as shown schematically in FIG. 4, they activate means for modifying a dose of NO, for example one or more virtual keys displayed by the graphical display means 4 having a touch panel, for example “+” or “−” keys, for incrementing or decrementing the value displayed by the graphical display means 4 in such a way as to modify the first dose of NO (D_{NO1}), in this case 20 ppmv for example, so as to obtain or fix the desired second dose of NO (D_{NO2}), in this case 16 ppmv for example.

Once the second dose of NO (D_{NO2}) has been fixed, they can confirm this dose using a confirmation key 51 for example, which is activated by the user pressing it with their finger.

According to another embodiment (not shown), the dose or doses of NO could also be input directly using a virtual or actual numerical keypad of the graphical user interface (GUI) of the apparatus or could be selected from a list of possible selectable values.

Therefore, according to the invention, the operating means 210 are configured to automatically calculate, on the basis of said second dose of NO (D_{NO2}), i.e. in this case 16 ppmv, second upper and lower NO alarm thresholds (Tₘₐₓ₂, Tₘᵢₙ₂) different to the first upper and lower NO alarm thresholds (Tₘₐₓ₁, Tₘᵢₙ₁), corresponding to second maximum (Tₘₐₓ₂) and minimum (Tₘᵢₙ₂) NO contents, wherein: Tₘₐₓ₂>D_{NO2}>Tₘᵢₙ₂.

These second upper and lower NO alarm thresholds (Tₘₐₓ₂, Tₘᵢₙ₂) are calculated like the first upper and lower NO alarm thresholds (Tₘₐₓ₁, Tₘᵢₙ₁), as explained above, and with the same tolerance value, namely 10 to 20%, preferably 20%.

Therefore, with a second dose of NO (D_{NO2}) set at 16 ppmv (with a first dose of NO (D_{NO1}) previously set at 20 ppmv) and a tolerance fixed at 20%, the apparatus 1 can automatically calculate second upper and lower NO alarm thresholds at 13 ppmv for the second minimum NO content (Tₘᵢₙ₂) and at 19 ppmv for the second maximum NO content (Tₘₐₓ₂), in other words second upper and lower thresholds at 16 ppmv+/−20% (i.e. +/−3 ppmv).

However, as previously, for doses of NO below 10 ppmv, the operating means 210 are configured to calculate and/or fix second maximum and minimum NO contents (Tₘₐₓ₂, Tₘᵢₙ₂) that preferably differ by at least 2 ppmv from the second dose of NO (D_{NO2}) set, preferably by +/−2 ppmv.

As explained above, keeping to a minimum difference of at least 2 ppmv relative to the dose of NO (D_{NO}) set, preferably a difference equal to +/−2 ppmv, for a dose of below 10 ppmv, makes it possible to prevent untimely (too frequent) triggering of alarms, while ensuring good patient safety, in particular for paediatric patients, typically newborns, babies, young children or the like, who have to receive lower doses of NO than adult patients.

As will be appreciated therefore, in this case, strict control of the alarm thresholds in the event of modification of the dose dispensed is essential to avoid dosage problems while ensuring that the treatment with NO that is administered to the patient is delivered effectively.

Since, in the context of the invention, the recalculation of the alarm thresholds in the event of modification of the dose of NO to be administered is performed automatically and instantaneously, in other words in real time, upon confirmation of the new dose of NO to be administered, in other words the second dose of NO (D_{NO2}), it is essential that the operating means can determine whether the new dose is below 10 ppmv and, if it is, act accordingly by calculating the new alarm thresholds while complying with the abovementioned condition, namely a difference of +/−2 ppmv with respect to the new dose selected by the user, i.e. the doctor or the like.

The fact that it is the apparatus that performs this recalculation makes it possible to avoid inputting errors or the like, which further improves patient safety.

It is particularly important to keep to this difference of at least 2 ppmv for doses of NO below 10 ppmv, preferably +/−2 ppmv, when doses are recalculated because it is at this moment that the new dose of NO selected is likely to fall below 10 ppmv, typically when the healthcare personnel notices that the initial dose is too high. To be specific, the starting dose selected in many hospital departments is generally at least 10 ppmv, for example 10, 15 or 20 ppmv, and the calculation of the upper and lower thresholds is thus performed at +/−10% to 20% as explained above.

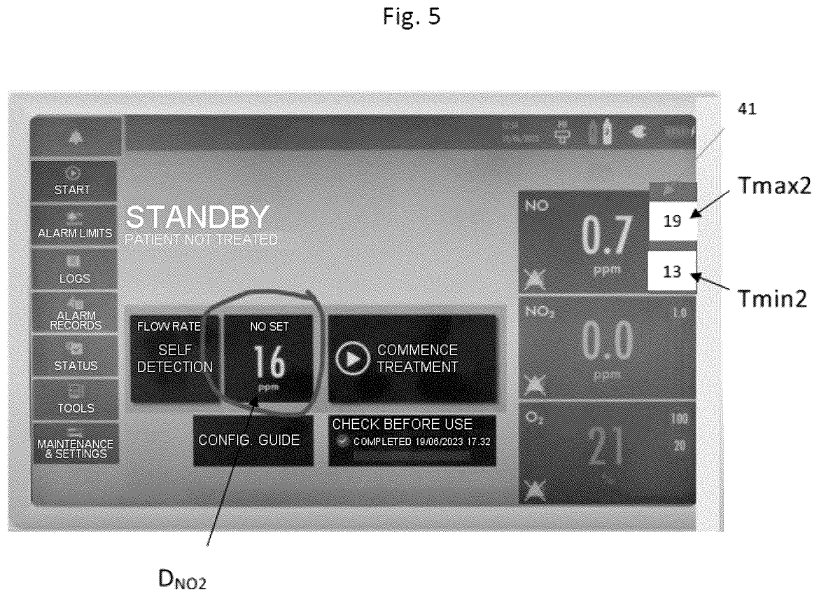

Then, as shown in FIG. 5, the displays shown on the graphical display means 4 change, in other words they are updated, so as to display not only the “new” or second dose of NO (D_{NO2}) but also the “new” or second maximum and minimum NO contents (Tₘᵢₙ₂, Tₘₐₓ₂) corresponding to the second upper and lower NO alarm thresholds that have been determined automatically on the basis of the second dose of NO (D_{NO2}) selected by the user.

Of course, if the user were to again change the dosage of NO, in other words apply a third dose of NO (or even further doses of NO, for example a fourth, a fifth, etc.), then the apparatus 1 would function in the same way to calculate “new” or third (or more) maximum and minimum NO contents (Tₘᵢₙ₃, Tₘₐₓ₃).

In all cases, the fact that the apparatus 1 is programmed to automatically update the alarm thresholds in the event of a change in the dose of NO, in other words the dosage, is advantageous because it prevents untimely triggering of alarms which could go off if the user were to forget to adapt the alarm thresholds accordingly or if they were to make a mistake in calculating the new alarm thresholds and were thus to enter incorrect threshold values.

Then, during operation of the apparatus 1, when the operating means 210 detect a concentration of NO in the combined NO/O₂/N₂ mixture which is higher than the first maximum NO content (Tₘₐₓ₁) or, in the event of a change in NO content, higher than a second maximum NO content (Tₘₐₓ₂), in other words exceeding the upper threshold (Tₘₐₓ₁, Tₘₐₓ₂), or, conversely, which is lower than the first minimum NO content (Tₘᵢₙ₁), or, in the event of a change in NO content, lower than a second minimum NO content (Tₘᵢₙ₂), in other words exceeding the lower threshold (Tₘᵢₙ₁, Tₘᵢₙ₂), they act on alarm means of the apparatus 1 to trigger an alarm, which may be acoustic, for example a sound coming from a buzzer or the like, and/or visual, for example a warning message displayed on the graphical display means 4 in order to warn the healthcare personnel that the NO content of the combined gas mixture is too high or, conversely, too low.

Of course, other alarms may be triggered by the operating means 210 in the event of detection of an excessively high NO₂ content, for example exceeding 1 to 3 ppmv approximately, or an excessively low oxygen content, for example below 19 to 20 vol % approximately. These alarms may be factory set and/or optionally, may be modifiable by the user.

A gas administration installation 100 including an NO delivery apparatus 1 according to the invention comprising means for automatic modification of the alarm thresholds upon changes in dose, may be used to administer nitric oxide (NO), i.e. the final NO/O₂/N₂ mixture obtained, by inhalation to persons, i.e. patients, suffering from acute pulmonary arterial hypertension, in particular to dilate their pulmonary vessels and increase their oxygenation by improving pulmonary gas exchange, in particular to treat persistent pulmonary arterial hypertension of the newborn (PPHN), acute respiratory distress syndrome (ARDS) observed mainly in adults, or pulmonary hypertension (PH) in cardiac surgery in adults or children.

What is claimed is:

1. Apparatus (1) for delivering NO-containing gas, comprising:
means for setting a dose of NO (50, 51) configured to allow a user to set a first dose of NO (D_{NO1}) of between 1 and 80 ppmv, and
operating means (210) with a microprocessor (211), configured to determine on the basis of said first dose of NO set (D_{NO1}), first upper and lower alarm thresholds corresponding to a first maximum NO content (Tₘₐₓ₁) and to a first minimum NO content (Tₘᵢₙ₁), wherein:
Tₘₐₓ₁>D_{NO1}>Tₘᵢₙ₁, and
means for modifying a dose of NO (50, 51) configured to allow a user to modify or adjust the first dose of NO (D_{NO1}) so as to obtain or fix a second dose of NO (D_{NO2}) that is different to the first dose of NO (D_{NO1}), and
characterized in that the operating means (210) are configured to automatically determine, on the basis of said second dose of NO (D_{NO2}), second upper and lower NO alarm thresholds that are different to said first upper and lower NO alarm thresholds, corresponding to a second maximum NO content (Tₘₐₓ₂) and to a second minimum NO content (Tₘᵢₙ₂), wherein: 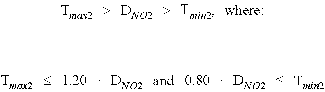
and, when the second dose of NO (D_{NO2}) is below 10 ppmv, wherein: Tₘₐₓ₂−D_{NO2}≥2 ppmv and D_{NO2}−Tₘᵢₙ₂≥2 ppmv.

2. Apparatus according to claim 1, characterized in that the operating means (210) are further configured to calculate and/or fix first maximum and minimum NO contents (Tₘₐₓ₁, Tₘᵢₙ₁) such that: 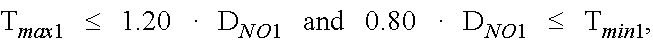
and wherein: Tₘₐₓ₁−D_{NO1}>2 ppmv and D_{NO1}−Tₘᵢₙ₁≥2 ppmv, when the dose of NO (D_{NO}) set is below 10 ppmv.

3. Apparatus according to claim 1, characterized in that it further comprises a graphical display means (4) configured to display at least:
the first dose of NO set (D_{NO1}) and the first maximum and minimum NO contents (Tₘᵢₙ₁, Tₘₐₓ₁) corresponding to the first upper and lower alarm thresholds, or
the second dose of NO (D_{NO2}) and the second maximum and minimum NO contents (Tₘᵢₙ₂, Tₘₐₓ₂) corresponding to the second upper and lower NO alarm thresholds that were determined.

4. Apparatus according to claim 1, characterized in that the operating means (210) are configured to determine the second minimum and maximum NO contents (Tₘᵢₙ₂, Tₘₐₓ₂) such that:
when: D_{NO2}<D_{NO1} then: Tₘₐₓ₂<Tₘₐₓ₁ and Tₘᵢₙ₂<Tₘᵢₙ₁, Or
when: D_{NO2}>D_{NO1} then: Tₘₐₓ₂>Tₘₐₓ₁ and Tₘᵢₙ₂>Tₘᵢₙ₁.

5. Apparatus according to claim 1, characterized in that it further comprises an internal gas circuit (200) for conveying a flow of NO-containing gas, comprising flow rate control means (220, 221) configured to control the flow of NO-containing gas within said gas circuit (200).

6. Apparatus according to claim 5, characterized in that the operating means (210) with a microprocessor (211) are configured to manage at least the flow rate control means (220, 221) to control the supply of NO-containing gas as a function of at least said first dose of NO set (D_{NO1}) or the second dose of NO (D_{NO2}) set.

7. Apparatus according to claim 5, characterized in that:
the internal gas circuit (200) of the apparatus (1) conveying the NO-containing gas is fluidically connected to a respiratory circuit (20; 21) conveying a respiratory gas containing oxygen, in order to inject the NO-containing gas into it and obtain a combined gas mixture containing NO and oxygen,
and the operating means (210) are configured to trigger an alarm by activating alarm means, when the NO content in the combined gas mixture is higher than or equal to the first or, depending on the circumstances, the second maximum NO content (Tₘₐₓ₁, Tₘₐₓ₂) or lower than or equal to the first or, depending on the circumstances, the second minimum NO content (Tₘᵢₙ₁, Tₘᵢₙ₂).

8. Apparatus according to claim 3, characterized in that NO concentration measurement means are arranged in such a way as to determine the NO content in the combined gas mixture within the internal gas circuit (200) and the operating means (210) are configured to manage a display (41), on the graphical display means (4), of the NO content (NOᵢₙₛₜ) in the combined gas mixture that has been determined by NO concentration measurement means.

9. Apparatus according to claim 1, characterized in that the means for setting a dose of NO or for modifying a dose of NO (50, 51) comprise one or more virtual keys displayed on the graphical display screen (4).

10. Apparatus according to claim 1, characterized in that, when the second dose of NO (D_{NO2}) is below 10 ppmv, the operating means (210) are configured to automatically determine the second upper and lower NO alarm thresholds corresponding to the second maximum and minimum NO contents (Tₘₐₓ₂, Tₘᵢₙ₂), such that: Tₘₐₓ₂=D_{NO2}+2 ppmv and Tₘᵢₙ₂=D_{NO2}−2 ppm.

11. Apparatus according to claim 1, characterized in that the second dose of NO (D_{NO2}) is between 1 and 80 ppmv.

12. Installation (100) for supplying NO-containing gas, comprising the NO delivery apparatus (1) according to claim 1, fed with an NO/N₂ gas mixture by an NO/N₂ mixture source, and a medical ventilator (50) configured to supply a flow of respiratory gas containing O₂.

13. Installation according to claim 12, characterized in that the medical ventilator (50) is configured to supply a flow of respiratory gas containing at least 20 vol % approximately of O₂, typically an NO/N₂ gas mixture or air.

14. Installation according to claim 12, characterized in that the NO delivery apparatus (1) and the medical ventilator (50) are fluidically connected to the respiratory circuit (20; 21) in order to supply it with gas flows.

15. Installation according to claim 12, characterized in that a flow rate sensor (25) is arranged in the respiratory circuit (20) between the medical ventilator (50) and an injection device (24).
